(19) United States
(12) Patent Application Publication
(10) Pub. No.: US 2024/0264088 A1
(43) Pub. Date: Aug. 8, 2024

(54) HYPERSPECTRAL IMAGING AND ARTIFICIAL INTELLIGENCE DETECTION METHODS AND SYSTEMS

(71) Applicants: SHWETA GUPTA, MILIPITAS, CA (US); ABHISHEK GOYAL, MILIPITAS, CA (US)

(72) Inventors: SHWETA GUPTA, MILIPITAS, CA (US); ABHISHEK GOYAL, MILIPITAS, CA (US)

(21) Appl. No.: 18/230,129

(22) Filed: Aug. 3, 2023

Related U.S. Application Data
(60) Provisional application No. 63/443,706, filed on Feb. 6, 2023.

(51) Int. Cl. G01N 21/88; G01N 21/3563; G01N 21/93; G01N 21/94; G01N 33/02; G01N 33/10; G06V 10/58

(52) U.S. Cl. CPC G01N 21/8851; G01N 21/3563; G01N 21/8806; G01N 21/93; G01N 21/94; G01N 33/025; G01N 33/10; G06V 10/58; G01N 2021/8845; G01N 2021/8887

(57) ABSTRACT

In one aspect, a computerized method for measuring a toxin in a food-commodity sample, comprising: implementing a hyperspectral imaging of the food-commodity sample with a hyperspectral digital camera; obtaining the hyperspectral image of the food-commodity sample; with at least one machine-learned toxin-detection model, implementing an AI analysis of hyperspectral image of food-commodity sample and determining a presence of the toxin in the food-commodity sample; and outputting a presence of the toxin in the food-commodity sample via a human-computer interface.

### CLAIM OF PRIORITY

This application claims priority to U.S. Provisional Patent Application No. 63/443,706, filed on 6 Feb. 2023 and titled HYPERSPECTRAL IMAGING AND ARTIFICIAL INTELLIGENCE DETECTION METHODS AND SYSTEMS. This Provisional Patent Application is hereby incorporated by reference in its entirety.

### BACKGROUND

Mycotoxin is an expanding threat to the food safety globally. Mycotoxins are a big threat to food safety. Mycotoxin contamination rate is growing globally. It refers to the toxins that are produced by fungi and are harmful to the crops. It can be found in a variety of food commodities. However, cereal grains, including corn, are among the most commonly contaminated crops, owing to improper management of crops during development, harvest and post-harvest processing. The United States is the largest producer of corn in the world, responsible for almost 40% of the world's total corn production. Hence, the corn contaminated with mycotoxins has a direct economic impact along with the adverse effects on the human and animal health. Various types of mycotoxins can be present in corn, which are produced by different fungi and molds. Among the various toxins, Aflatoxins (AFLA), Deoxynivalenol (DON), Zearalenone (ZEA), and Fumonisin (FUM) are the most important mycotoxins in corn.

For example, at the beginning of 2021, there was a broad recall of products that contained high levels of aflatoxin. This mycotoxin is known to develop on corn and other grains used in the preparation of pet food formulations. Midwestern Pet Food, Inc. produced Sportmix pet food, which was consumed by over 220 sickened pets and led to the death of more than 130 pets in the United States.

Currently, the conventional standard methods widely employed for the mycotoxin detection and analysis are high performance liquid chromatography (HPLC), thin layer chromatography (TLC), Enzyme-Linked immunosorbent assay (ELISA), liquid chromatography coupled with mass spectrometry (LC-MS), and Lateral flow Immunoassay. These detection methods are accurate and sensitive; however, they possess a number of limitations. They are labor-intensive, destructive in nature, need safety measures, require trained operators, are slow in examination of samples, and are not cost-effective, which limits their feasibility for routine use.

Due to these limitations, the food industry is also not able to scale up their testing for every incoming corn-loaded truck that leads to significant mycotoxin levels in the finished products. Thus, there is a need of rapid, safe, efficient and reliable methods for the detection of mycotoxins in corn, and other grains.

To overcome the aforementioned drawbacks, several efficient spectroscopy-based mycotoxin detection and classification methods have been investigated over the years, such as near infrared spectroscopy, Raman spectroscopy, hyperspectral imaging, and Fourier transform infrared spectroscopy. Hyperspectral imaging techniques have been of particular interest as they are faster, non-destructive, and safer. Sampling methods employed in near infrared spectroscopy can result in uncertainties, whereas hyperspectral imaging scans and analyzes a significant part of a load, reducing the sampling error. Near-infrared (NIR) spectral data is often intricate, with overlapping and feeble absorption bands leading to complex spectral outcomes. This necessitates the use of analytical tools to address the issue. Raman, Vis/NIR, Fluorescence and SWIR HSI have been studied for aflatoxin detection in Maize, SWIR HSI for aflatoxin examination on corn kernels, Vis/NIR HSI to assess Fusarium infection on wheat kernels and flour, and HSI for DON screening in wheat kernels.

These techniques have shown promising results. However, HSI methods have not been reported yet for the assessment of all the other major significant mycotoxins on corn. In addition, these investigations involve analytical algorithms, such as linear discriminant analysis (LDA), linear support vector machines (LSVM), quadratic discriminant analysis (QDA), and quadratic support vector machines (QSVM) algorithms, principal component analysis (PCA), and partial least squares discriminant analysis (PLS-DA) model for further classification or analysis of massive HSI data. Hence, there is a need to develop a novel and fast method to predict Mycotoxins at these LoD.

### SUMMARY OF THE INVENTION

In one aspect, a computerized method for measuring a toxin in a food-commodity sample, comprising: implementing a hyperspectral imaging of the food-commodity sample with a hyperspectral digital camera; obtaining the hyperspectral image of the food-commodity sample; with at least one machine-learned toxin-detection model, implementing an AI analysis of hyperspectral image of food-commodity sample and determining a presence of the toxin in the food-commodity sample; and outputting a presence of the toxin in the food-commodity sample via a human-computer interface.

### BRIEF DESCRIPTION OF THE DRAWINGS

The present application can be best understood by reference to the following description taken in conjunction with the accompanying figures, in which like parts may be referred to by like numerals.

FIG. 1 illustrates an example process for measuring mycotoxin in corn, according to some embodiments.

FIG. 2 illustrates an example process for developing and using an AI model(s) for the real time mycotoxin detection in corn, according to some embodiments.

FIG. 3 an example workflow for detecting mycotoxin in corn kernels, according to some embodiments.

FIG. 4-6 illustrates example images of a hyperspectral imaging system obtaining one or more digital hyperspectral images of corn-kernel samples, according to some embodiments.

FIG. 7-9 illustrates an example set of screenshots showing the use of an AI model(s) on the one or more digital hyperspectral images of corn-kernel samples, according to some embodiments.

FIG. 10 depicts an exemplary computing system that can be configured to perform any one of the processes provided herein.

FIG. 11 illustrates an example process for using Artificial intelligence (AI) to detect mycotoxins, according to some embodiments.

FIG. 12 illustrates a table demonstrating example action levels that can be used for different mycotoxins for corn according to FDA regulations, according to some embodiments.

FIG. 13 illustrates an example process for hyperspectral imaging and artificial intelligence detection, according to some embodiments.

FIG. 14 illustrates an example spectrum of four randomly selected corn samples, according to some embodiments.

FIG. 15 illustrates an example process for HSI processing, according to some embodiments.

FIG. 16 illustrates an example screenshot, according to some embodiments.

The Figures described above are a representative set and are not an exhaustive with respect to embodying the invention.

### DESCRIPTION

Disclosed are a system, method, and article of manufacture for hyperspectral imaging and artificial intelligence detection methods and systems. The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments.

Reference throughout this specification to “one embodiment,” “an embodiment,” “one example,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown. It is noted that use of the term ‘real time’ assumes various latencies such as networking latencies, computer processing latencies, etc.

### Definitions

The following terminology is used in example embodiments:

Aflatoxins are various poisonous carcinogens and mutagens that are produced by certain molds, particularly Aspergillus species. The fungi grow in soil, decaying vegetation and various staple foodstuffs and commodities such as hay, sweetcorn, wheat, millet, sorghum, cassava, rice, chili peppers, cottonseed, peanuts, tree nuts, sesame seeds, sunflower seeds, and various spices.

Computer vision is an interdisciplinary scientific field that deals with how computers can gain high-level understanding from digital images or videos.

Fumonisins are a group of mycotoxins derived from Fusarium and their Liseola section.

Hyperspectral imaging collects and processes information from across the electromagnetic spectrum. Hyperspectral imaging can obtain the spectrum for each pixel in the image of a scene, with the purpose of finding objects, identifying materials, or detecting processes. Hyperspectral imaging can include push broom scanners and related whisk broom scanners (e.g. spatial scanning). These can read images over time. Hyperspectral imaging can include band sequential scanners (e.g. spectral scanning), which acquire images of an area at different wavelengths. Hyperspectral imaging can include snapshot hyperspectral imaging. This can use a staring array to generate an image in an instant.

Limit of detection (LOD) is the lowest signal, or the lowest corresponding quantity to be determined (and/or extracted) from the signal, that can be observed with a sufficient degree of confidence or statistical significance.

Mycotoxin is a toxic secondary metabolite produced by organisms of kingdom Fungi and is capable of causing disease and death in both humans and other animals. Mycotoxin can include the toxic chemical products produced by fungi that colonize crops (e.g. corn, etc.).

Silage is a type of fodder made from green foliage crops which have been preserved by fermentation to the point of acidification.

Vomitoxin, also known as deoxynivalenol (DON), is a type B trichothecene, an epoxy-sesquiterpenoid. This mycotoxin occurs predominantly in grains such as wheat, barley, oats, rye, and corn, and less often in rice, sorghum, and triticale. The occurrence of deoxynivalenol is associated primarily with Fusarium graminearum (Gibberella zeae) and F. culmorum, both of which are important plant pathogens which cause Fusarium head blight in wheat and gibberella or Fusarium ear blight in corn.

Zearalenone (ZEN), also known as RAL and F-2 mycotoxin, is a potent estrogenic metabolite produced by some Fusarium and Gibberella species. The Gibberella zeae, the fungal species where zearalenone was initially detected, in its asexual/anamorph stage is known as Fusarium graminearum. Several Fusarium species produce toxic substances of considerable concern to livestock and poultry producers, namely deoxynivalenol, T-2 toxin, HT-2 toxin, diacetoxyscirpenol (DAS) and zearalenone.

These definitions are provided by way of example and not of limitation.

### Example Systems and Methods

An example embodiment relates to a method and a system for hyperspectral imaging and artificial intelligence detection methods and systems. It is noted that mycotoxin and corn are provided by way of example. In other embodiments, other toxins/toxin sources can be identified. For example, other toxins can include, inter alia: T2, HT2, total T2 HT2, Fumonisin, Vomitoxin, Aflatoxin, Zearalenone, etc. These can be identified on other types of grains, seeds, plant products, silage (e.g. corn silage, etc.), food commodities, or by products such as DDGS, Corn Gluten Meal, wheat midds, Corn Silage, TMR (Total Mixed Ration), Peanuts, etc. In some example embodiments, the presence of vitamins can be identified in the premix.

It is noted that AI models and/or methods used herein can be used to determine/detect/measure/predict other attributes of object of analysis, such as: vitamin content (e.g. vitamin A, vitamin D and vitamin E), mineral content, grain damage (e.g. extent of damage, type of damage, etc.), other grain attributes, particle size(s) (e.g. particle size of corn grain, etc.), other physical parameters, various chemical parameters, various biochemical parameters, pathogen-related parameters, etc. AI models can be developed and used for other food commodities (e.g. raw state, finished state, etc.). It is noted that AI systems used herein can be extended using a smart phone and/or portable devices. For example, methods and systems provided herein can be implemented in the form of a smart phone application. It is noted that methods and systems provided herein can be implemented with respect to pharmaceuticals products, soil samples, geological samples, dietary supplements, water supply, laboratory samples, cosmetics, other organic and/or non-organic compounds, etc.

In one example, Hyperspectral imaging (HSI) is a combination of digital imaging and spectroscopy. It is a cost-effective method with notable benefits that can be utilized for evaluating fungi and mycotoxins. HSI analysis is non-destructive, rapid, and generates spectral data for every pixel location. This technique has the potential to substitute expensive and time-consuming procedures for analyzing mycotoxins in grains, and can play a crucial role in identifying contaminated grains Hyperspectral imaging enables the comprehensive analysis of a sample's spectral (e.g. spectroscopic) and spatial (e.g. imaging) features by capturing the complete spectral response at various wavelengths, such as UV-visible, visible-NIR, shortwave infrared (SWIR), or thermal infrared, within each pixel of the hyperspectral image. Sensors (e.g., a hyperspectral digital camera, etc.) can be used to obtain a hyperspectral image of a corn sample. This can be a 3D image that captures more than 200 spectral bands at each and every pixel.

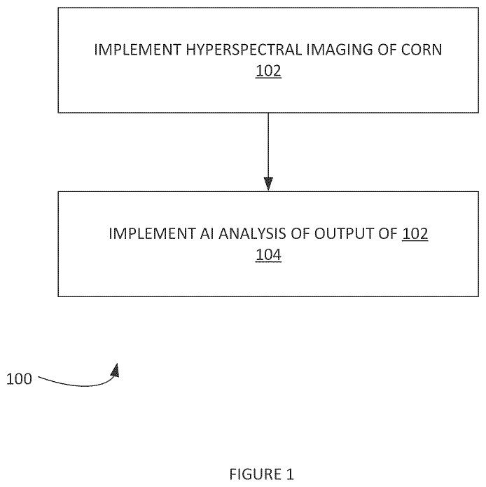

FIG. 1 illustrates an example process 100 for measuring mycotoxin in corn, according to some embodiments. In step 102, process 100 can implement hyperspectral imaging of corn. Sensors (e.g. a hyperspectral digital camera, etc.) can be used to obtain a hyperspectral image of a corn sample. This can be a 3D image that captures more than 200 spectral bands at each and every pixel.

In step 104, implement AI analysis of output of 102 to determine the presence of mycotoxin in corn. Step 104 can identify N-types (e.g. four types) mycotoxins. For each type of mycotoxin, an AI/ML model can be trained and validated. AI/ML model training and validation can be a combination of classical machine learning and deep learning techniques (e.g. Neural Network based deep learning techniques, etc.). In one example, four AI models developed and provided one AI model for each mycotoxin such as: ZEA, DON (Vomitoxin), Aflatoxin, Fumonisin. However, in other example embodiments, other AI models can be developed for other types of mycotoxin and/or other toxin sources. Each AI model can include a Limit of Detection (LoD) for each of these mycotoxins. Each AI model can be quantitative and/or qualitative. For example, the output can include quantitative information.

Step 104 can utilize machine learning methods and systems. Step 104 can utilize one or more machine learning process(es). Machine learning process(es) can manage and implement the various machine learning operations discussed herein. Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity, and metric learning, and/or sparse dictionary learning. Random forests (RF) (e.g. random decision forests) are an ensemble learning method for classification, regression, and other tasks, which operate by constructing a multitude of decision trees at training time and outputting the class that is the mode of the classes (e.g. classification) or mean prediction (e.g. regression) of the individual trees. RFs can correct for decision trees' habit of overfitting to their training set. Deep learning is a family of machine learning methods based on learning data representations. Learning can be supervised, semi-supervised or unsupervised.

Machine learning can be used to study and construct algorithms that can learn from and make predictions on data. These algorithms can work by making data-driven predictions or decisions, through building a mathematical model from input data. The data used to build the final model usually comes from multiple datasets. In particular, three data sets are commonly used in different stages of the creation of the model. The model is initially fit on a training dataset, which is a set of examples used to fit the parameters (e.g. weights of connections between neurons in artificial neural networks) of the model. The model (e.g. a neural net or a naive Bayes classifier) is trained on the training dataset using a supervised learning method (e.g. gradient descent or stochastic gradient descent). In practice, the training dataset often consists of pairs of an input vector (or scalar) and the corresponding output vector (or scalar), which is commonly denoted as the target (or label). The current model is run with the training dataset and produces a result, which is then compared with the target, for each input vector in the training dataset. Based on the result of the comparison and the specific learning algorithm being used, the parameters of the model are adjusted. The model fitting can include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g. the number of hidden units in a neural network). Validation datasets can be used for regularization by early stopping: stop training when the error on the validation dataset increases, as this is a sign of overfitting to the training dataset. Finally, the test dataset is a dataset used to provide an unbiased evaluation of a final model fit on the training dataset. If the data in the test dataset has never been used in training (e.g. in cross-validation), the test dataset is also called a holdout dataset.

It is noted that ML modules can be utilized to perform and/or optimize the following functionalities: a selection of region of interest, preprocessing operations, noise removal operations, optimal band selection, etc.

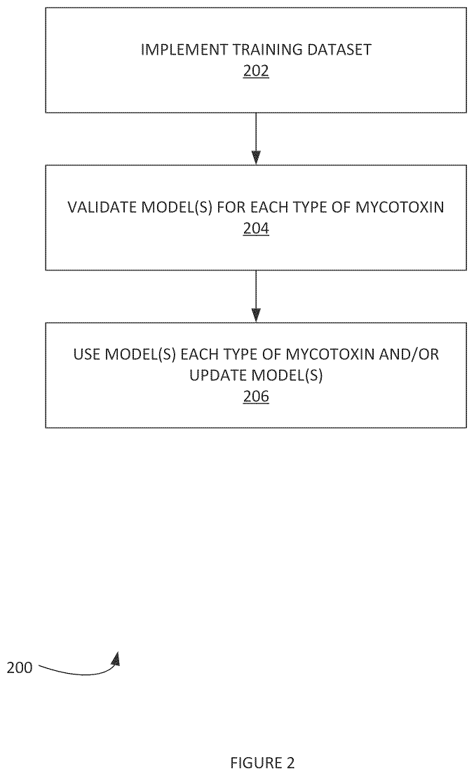

FIG. 2 illustrates an example process for developing and using an AI model(s) for the real time mycotoxin detection in corn, according to some embodiments. In step 202, process 200 can implement a training dataset. The training datasets can be a set of examples used to fit the parameters of the AI model. The training data set can include a library of hyperspectral images of the object of analysis (e.g. a library of hyperspectral images of corn that includes hyperspectral images of corn that is infected by mycotoxin and corn that is not infected by mycotoxin, etc.). The library of hyperspectral images can include hyperspectral images without the presence of the material being tested for detection and hyperspectral images of the materials with the presence of the material being tested for detection. The AI model can be trained on the training data set using various methods, including, inter alia: a supervised learning method, optimization methods (e.g. gradient descent, stochastic gradient descent, etc.).

In step 204, process 200 can validate AI model(s) for each type of mycotoxin. These AI models can be fitted models that are used to predict the responses for the observations in a second data set called the validation data set. The validation data set then provides an unbiased evaluation of a model fit on the training data set while tuning the model's hyperparameters. Validation datasets can be used for regularization (e.g. stopping training when the error on the validation data set increases, etc.).

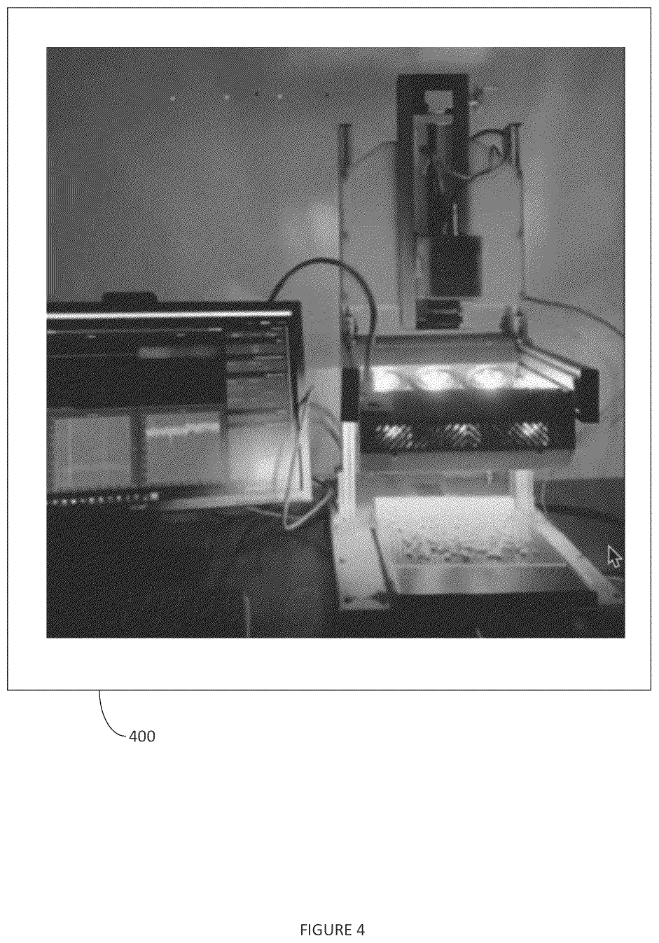

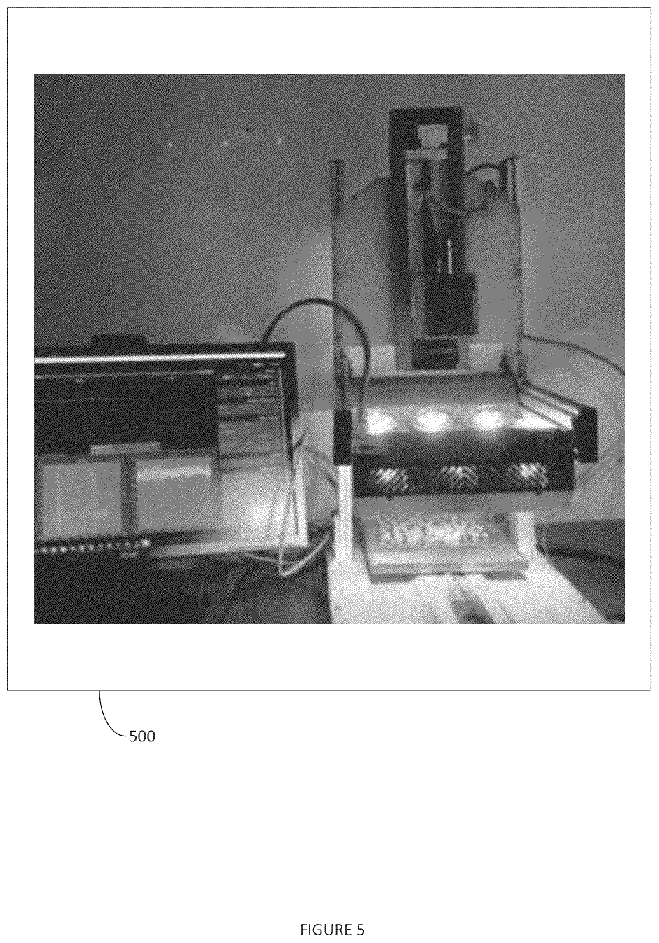

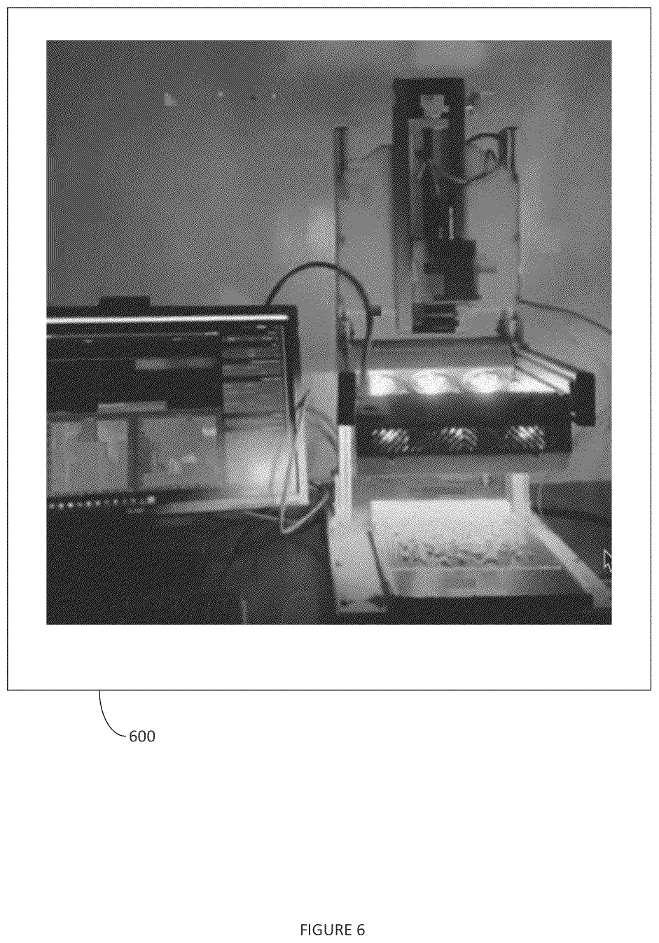

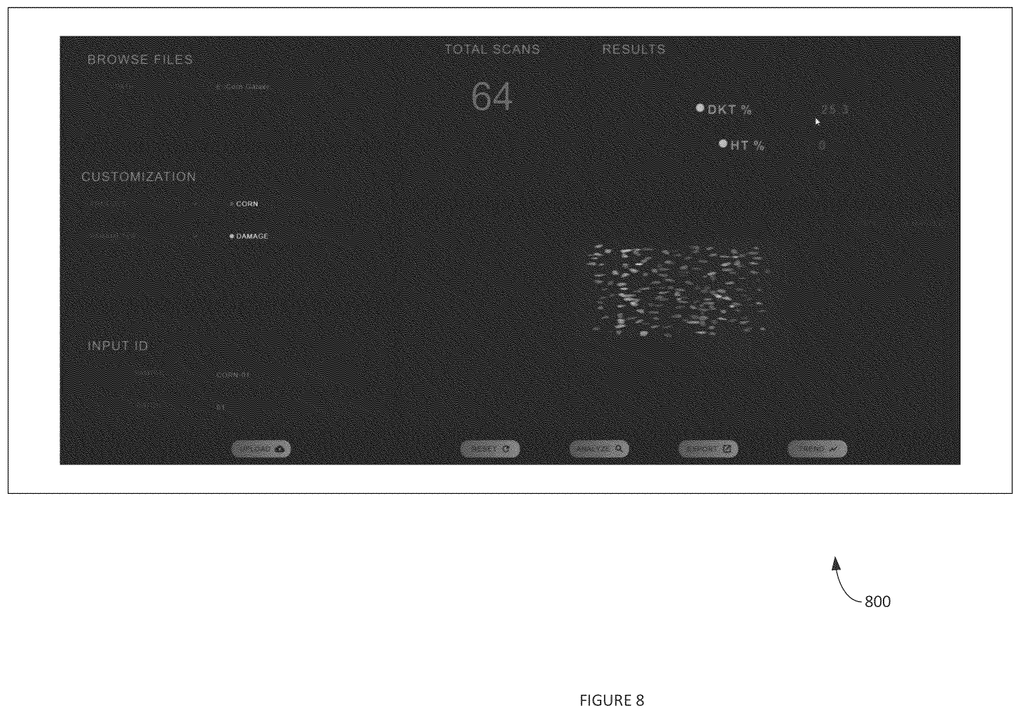

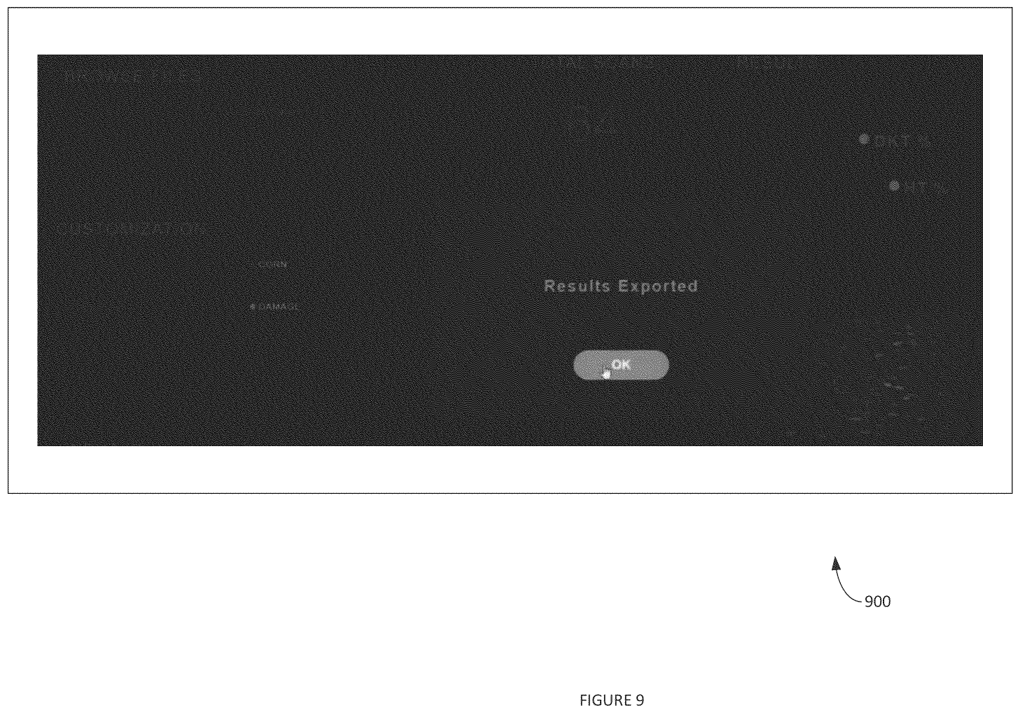

In step 206, process 200 can use the AI model(s) to identify each type of mycotoxin and/or update the current AI model(s). An example of this is provided in FIGS. 4-9 infra.

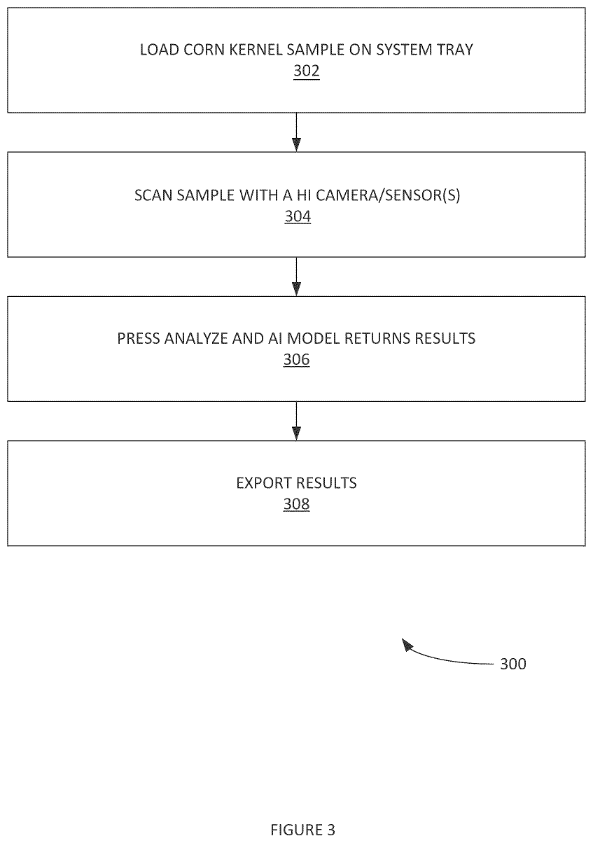

FIG. 3 an example workflow for detecting mycotoxin in corn kernels, according to some embodiments. In step 302, process 300 can load corn kernel samples on a system tray. In step 304, process 300 can scan the sample with a HSI camera/sensor(s). In step 306, process 300 can press analyze and the AI model returns results. In step 308, process 300 can export results (e.g. via an Excel spreadsheet, electronic mail, etc.). Again, an example of this is provided in FIGS. 4-9 infra.

FIG. 4-6 illustrates example images 400-600 of a hyperspectral imaging system obtaining one or more digital hyperspectral images of a corn-kernel sample, according to some embodiments.

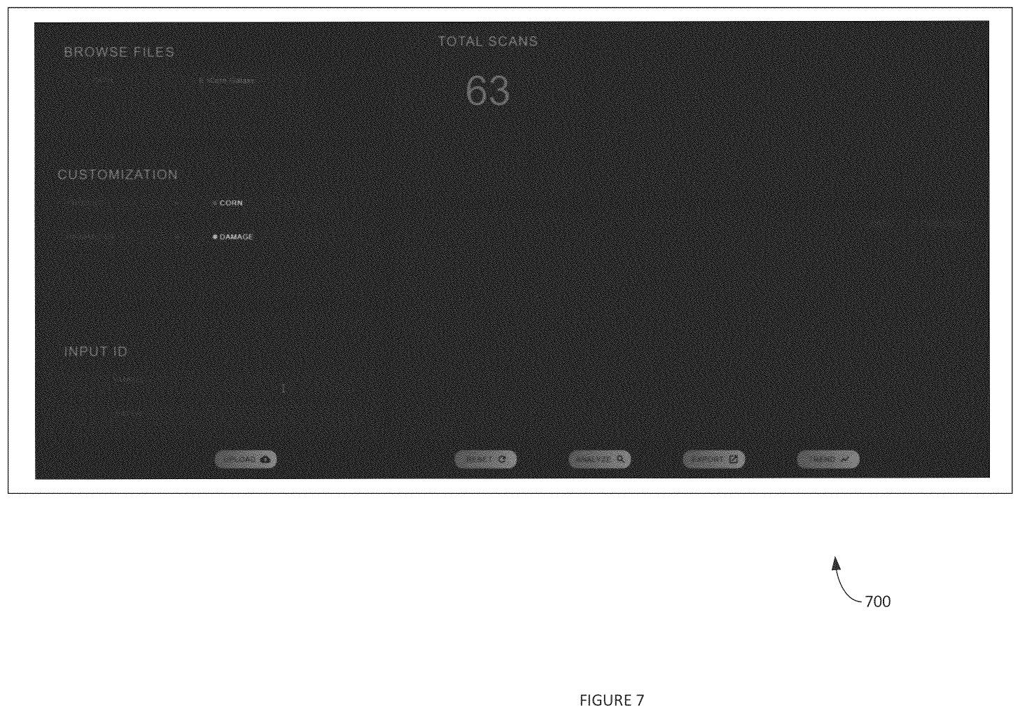

FIG. 7-9 illustrates an example set of screenshots 700-900 showing the use of an AI model(s) on the one or more digital hyperspectral images of a corn-kernel sample, according to some embodiments. As shown in screenshots 700-900, the sample data can be analyzed, viewed and exported (e.g. as described supra in process 300, etc.).

### Additional Computing Systems

Processes 100-300 can use various computing systems to be implemented. These can include, inter alia: hyperspectral imaging camera(s), lab scanner(s), high speed PC, etc.

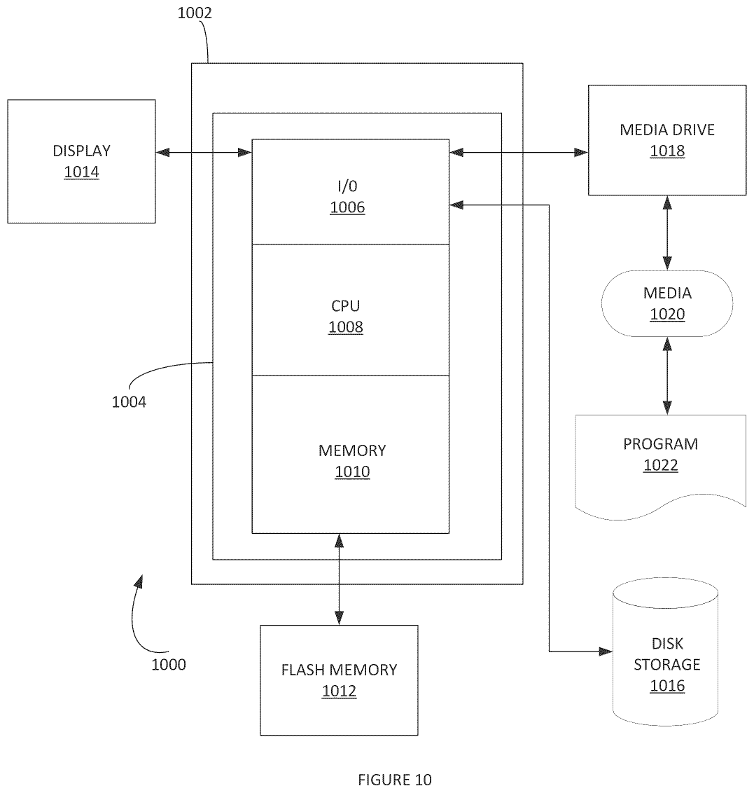

FIG. 10 depicts an exemplary computing system 1000 that can be configured to perform any one of the processes provided herein. In this context, computing system 1000 may include, for example, a processor, memory, storage, and I/O devices (e.g., monitor, keyboard, disk drive, Internet connection, etc.). However, computing system 1000 may include circuitry or other specialized hardware for carrying out some or all aspects of the processes. In some operational settings, computing system 1000 may be configured as a system that includes one or more units, each of which is configured to carry out some aspects of the processes either in software, hardware, or some combination thereof.

FIG. 10 depicts computing system 1000 with a number of components that may be used to perform any of the processes described herein. The main system 1002 includes a motherboard 1004 having an I/O section 1006, one or more central processing units (CPU) 1008 and/or graphical processing unit (GPU), and a memory section 1010, which may have a flash memory card 1012 related to it. The I/O section 1006 can be connected to a display 1014, a keyboard and/or another user input (not shown), a disk storage unit 1016, and a media drive unit 1018. The media drive unit 1018 can read/write a computer-readable medium 1020, which can contain programs 1022 and/or databases. Computing system 1000 can include a web browser. Moreover, it is noted that computing system 1000 can be configured to include additional systems in order to fulfill various functionalities. Computing system 1000 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol, etc.

### Additional Methods and Examples

FIG. 11 illustrates an example process for using Artificial intelligence (AI) to detect mycotoxins, according to some embodiments. In step 1102, process 1100 can analyze the hyperspectral cube in 900-1700 nm spectra of NIR for mycotoxin detection, since the NIR region is predominantly used for analyzing chemical compounds and water content.

In step 1104, process 100 can combine AI with HSI to predict mycotoxins in various commodities/grains. Process 1100 can be used to predict the four major mycotoxins (e.g. AFLA, DON, ZEA and FUM) in corn samples using Hyperspectral imaging and AI techniques for rapid screening purposes. This method is safe to use, non-destructive, faster, accurate, and inexpensive.

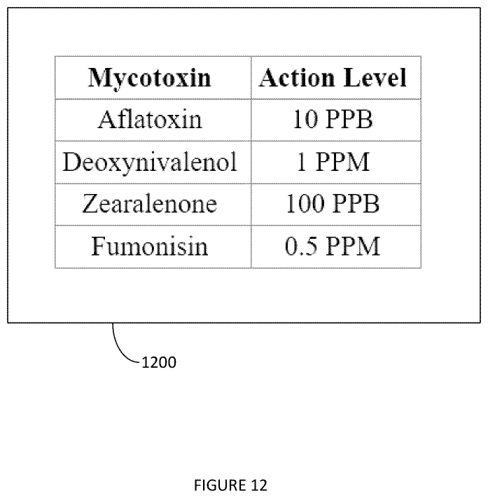

FIG. 12 illustrates a table 1200 demonstrating example action levels that can be used for different mycotoxins for corn according to FDA regulations, according to some embodiments. Action levels for different Mycotoxins for Corn are provided. It is noted that the present thresholds are provided by way of example and not of limitation. In other example embodiments, other threshold values can be utilized.

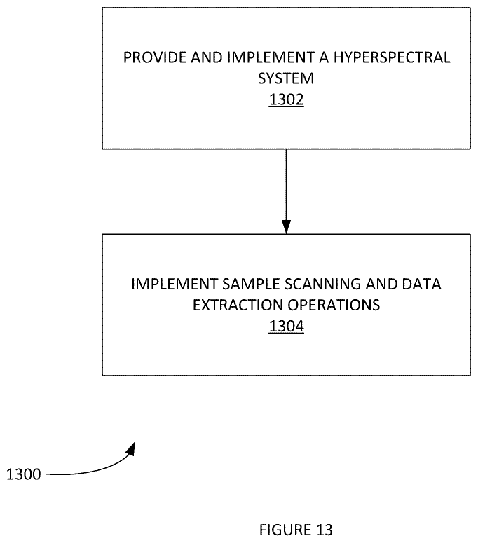

FIG. 13 illustrates an example process 1300 for hyperspectral imaging and artificial intelligence detection, according to some embodiments. In step 1302, process 1300 provides and implements a Hyperspectral System. The setup for capturing hyperspectral images of corn samples was established in a dark-lighting environment using push broom technology. In one example, the HSI system consists of five main components: a push broom camera for line-scanning the sample, an illumination unit comprising six 100 W tungsten halogen lamps to ensure sufficient lighting, a mobile tray controlled by a stepper motor for conveying the sample, a computer equipped with image acquisition software, and a white Teflon tile to use as a reference for hyperspectral image correction. In one example, the push broom camera includes an imaging spectrograph and a high-performance CCD camera paired with a camera lens. It is employed to gather comprehensive spectral and spatial information of the sample in a line-by-line manner within the near-infrared spectral range of 900-1700 nm. Each scanning process of the HSI system allows for capturing spectral information spanning hundreds of consecutive wavebands. These embodiments are provided by way of example and not of limitation.

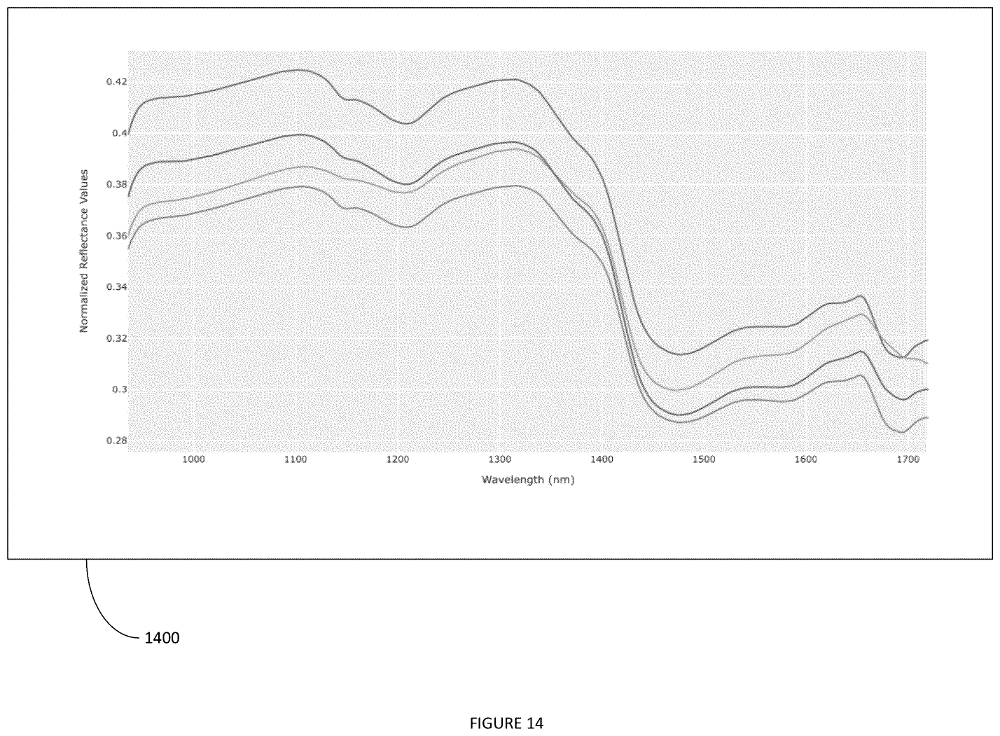

In one example embodiment, a total of 224 wavebands comprising n-number pixels can be acquired. Additionally, the entire system achieves a spectral resolution of 3.45 nm. Moreover, white and dark reference images were acquired to rectify the camera-generated noise. To obtain a white image (whiteREF), a white Teflon sheet reflecting almost 99% of the incident light was used, whereas for the dark image (darkREF), the camera was covered with a cap and the light was turned off. When converting hyperspectral data into reflectance, a white reference (white tile) is needed. It measures the incoming light seen by the hyperspectral camera, considering the optics' transmission and the detector's Quantum Efficiency. The role of white reference is crucial in obtaining good reflectance data with a hyperspectral instrument. The spectrums of white light and four randomly selected corn samples are shown in FIG. 14 infra, respectively for the range of 900-1700 nm. It is noted that different HSI ranges can be used in various other examples (e.g. 400-900 nm (VNIR), NIR (900-1700 nm), SWIR (900-2500 nm), etc.). Also, the mycotoxin test can use lower range HSI i.r. 400-900 nm in some examples. The spectrums display the variation in reflectance values with respect to the wavelength.

In step 1304, process 1300 implements sample scanning and data extraction operations. The samples were scanned with the help of hyperspectral imaging system in the NIR (near-infrared) range of 900-1700 nm.

FIG. 14 illustrates an example spectrum of four randomly selected corn samples, according to some embodiments. The spectrums display the variation in reflectance values with respect to the wavelength.

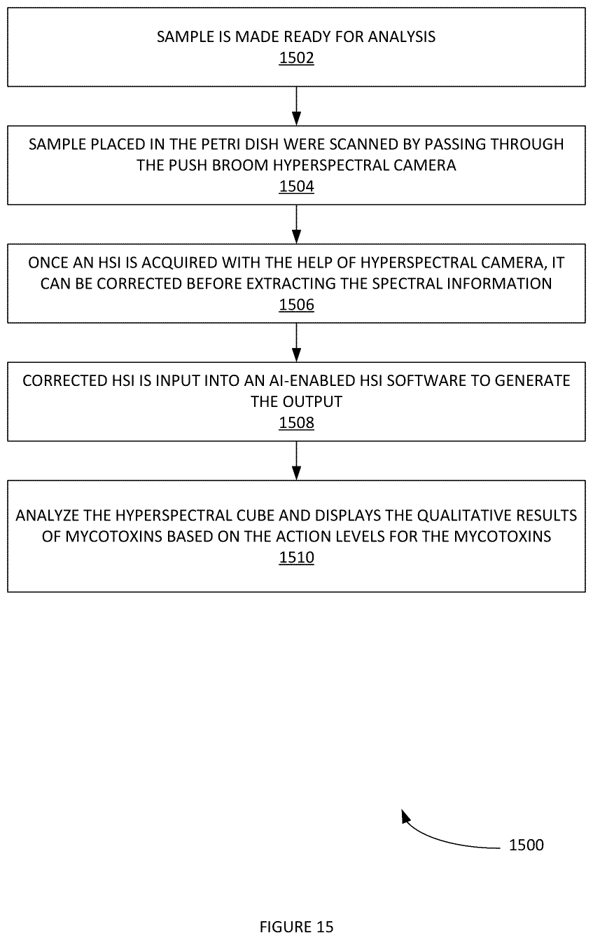

FIG. 15 illustrates an example process 1500 for HSI processing, according to some embodiments. In step 1502, the sample (e.g. corn, etc.) is made ready for analysis. In one example, the corn sample available in whole form is pulverized into a fine powder using a food grade grinder. Subsequently, the ground corn sample is placed in a petri dish measuring 10 cm*10 cm and filled until it reached the brim. AI analysis is agnostic to corn particle size.

In step 1504, the sample is placed in the petri dish were scanned by passing through the push broom hyperspectral camera. The process can take 10-15 seconds to record the sample and generate a hyperspectral data cube, 100-180 MB in size. Once an HSI is acquired with the help of hyperspectral camera, it can be corrected before extracting the spectral information in step 1506. The following equation is used for the correction of HSI:

$$Corrected H S I = ( H S I - darkREF ) / ( whiteREF - darkREF ) .$$

(1) Where darkREF and whiteREF indicate the dark and white reference images, respectively.

AI-enabled Hyperspectral imaging (HSI) and Output Generation is now discussed. The corrected HSI is input into an AI-enabled HSI software to generate the output in step 1508. The AI-enabled HSI software shows the corn mycotoxin parameters (e.g. AFLA, DON, ZEA, FUM) for which the analysis has to be conducted. Further, process 1500 analyzes the hyperspectral cube and displays the qualitative results of Mycotoxins within five (5) seconds based on the action levels for the mycotoxins in step 1510. The AI-enabled HSI software also shows the image of the corn sample used. No chemicals or reagents may be required to perform these tests. The qualitative results are shown in terms of the action levels for all the mycotoxins. The action levels considered for the mycotoxins are given in FIG. 12 supra. For instance, for a sample, the AI-enabled HSI software will present the results as AFLA<10 ppb, DON>1 ppm, FUM>0.5 ppm, and ZEA<100 ppb. It can indicate that the sample has higher concentrations of DON and FUM, whereas less concentrations of AFLA and ZEA mycotoxins.

In one example, the Hyperspectral Imaging system can include different optical sensors that can be used. It can have a range is 900-1700 nm and can work using 400-900 nm and/or 900-2500 nm. It can include different sources of light (e.g. halogens, LEDs, etc.). Additionally, different types of hyperspectral imaging can be used (e.g. push broom, line scanning, etc.). Moreover, different hyperspectral cameras can be used (e.g. not specific to one manufacturer).

The GUI/Dashboard can be used to display results (e.g. with a web application, desktop application, mobile device application, etc.).

Process 1500 can use various prediction methodologies. These can be used in predicting mycotoxins in naturally occurring samples. These can include the mycotoxins discussed supra and can be extended to other mycotoxins. Example commodities can be grains such as corn, rice, wheat, barley and their byproducts such as corn silage, TMR, corn gluten meal, corn feed meal, soybean meal, DDGSs, etc. The grains can be whole kernels or ground. Mycotoxins can be naturally occurring or spiked (e.g. articulated). The measurements can also be qualitative and/or extended to quantitative results. AI systems can be used (e.g. pre-processing module, synthetic dataset generation module, feature extraction module, optimal band selection module, neural networks and/or non-neural network-based algorithms, etc.

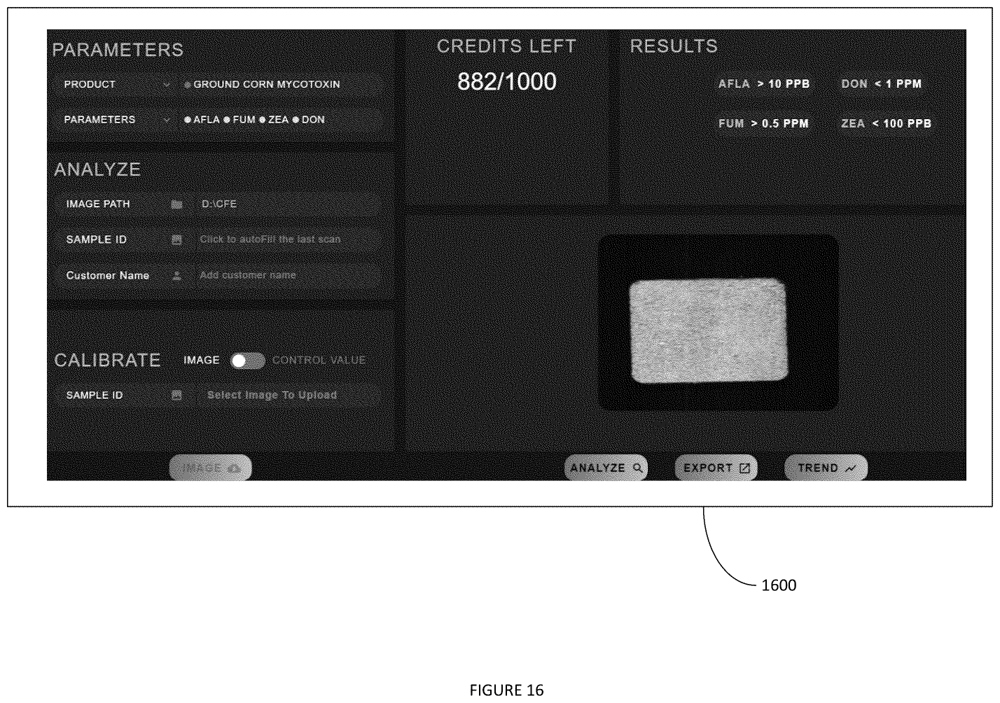

FIG. 16 illustrates an example screenshot 1600, according to some embodiments. In this particular example, the product of analysis is a Ground Corn Mycotoxin Parameter. The ML/AI models for all four (4) mycotoxins: Aflatoxin, ZEA, Fumonisin, DON are utilized for the analysis. A user can implement a search using the analyze button. As shown, the results are provided under the “Results” section with action levels for the mycotoxins. It is noted that the results can also be exported.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed by this United States patent:

1. A computerized method for measuring a toxin in a food-commodity sample, comprising:
implementing a hyperspectral imaging of the food-commodity sample with a hyperspectral digital camera;
obtaining the hyperspectral image of the food-commodity sample;
with at least one machine-learned toxin-detection model, implementing an AI analysis of hyperspectral image of food-commodity sample and determining a presence of the toxin in the food-commodity sample; and
outputting a presence of the toxin in the food-commodity sample via a human-computer interface.

2. The computerized method of claim 1, wherein the toxin comprises a mycotoxin.

3. The computerized method of claim 2, wherein the food commodity comprises a grain.

4. The computerized method of claim 3, wherein the grain comprises a corn-kernel sample.

5. The computerized method of claim 4, with at least one machine-learned toxin-detection model comprises four machine-learned mycotoxin detection models.

6. The computerized method of claim 5, wherein a first machine-learned model comprises a ZEA mycotoxin machine-learned that is trained and validated with a plurality of ZEA mycotoxin samples.

7. The computerized method of claim 6, wherein a second machine-learned model comprises a Fumonisin mycotoxin machine-learned that is trained and validated with a plurality of Fumonisin mycotoxin samples.

8. The computerized method of claim 7, wherein a third machine-learned model comprises a Vomitoxin mycotoxin machine-learned that is trained and validated with a plurality of Vomitoxin mycotoxin samples.

9. The computerized method of claim 8, wherein a fourth machine-learned model comprises an Aflatoxin mycotoxin machine-learned that is trained and validated with a plurality of Aflatoxin mycotoxin samples.

10. The computerized method of claim 9, further comprising:
generating a hyperspectral cube in a 900-1700 nm spectra of Near-infrared (NIR) as the hyperspectral image.

11. The computerized method of claim 10, further comprising:
for each of the four machine-learned mycotoxin detection models, outputting a corn mycotoxin parameter.

12. The computerized method of claim 11, wherein the corn mycotoxin parameter comprises a quantitative parameter or a qualitative parameter.

13. The computerized method of claim 12, wherein once the hyperspectral image is acquired form a hyperspectral camera:
before extracting the spectral information from the hyperspectral image, correcting the hyperspectral image.

14. The computerized method of claim 13, wherein the hyperspectral image is corrected using an equation comprising:
$$H S I = ( H S I - darkREF ) / ( whiteREF - darkREF ) .$$

15. The computerized method of claim 13, wherein the darkREF indicates a dark reference image and wherein the whiteREF indicates a white reference image.

16. The computerized method of claim 5, further comprising:
using the hyperspectral cube as an input for the at least one machine-learned toxin-detection model.

17. A computerized method for measuring a content in a food-commodity sample, comprising:
implementing a hyperspectral imaging of the food-commodity sample with a hyperspectral digital camera;
obtaining the hyperspectral image of the food-commodity sample;
with at least one machine-learned content-detection model, implementing an AI analysis of hyperspectral image of food-commodity sample and determining a presence of the content in the food-commodity sample; and
outputting a presence of the content in the food-commodity sample via a human-computer interface.

18. The computerized method of claim 17, wherein the content comprises a vitamin content in the food-commodity sample.

19. The computerized method of claim 17, wherein the content comprises a mycotoxin.

20. The computerized method of claim 17, wherein the food-commodity sample comprises a corn sample, and wherein the content comprises a particle size of the corn sample.
